(19) United States
(12) Patent Application Publication
(10) Pub. No.: US 2021/0388345 A1
(43) Pub. Date: Dec. 16, 2021

(54) PROFILING OF RHEUMATOID ARTHRITIS AUTOANTIBODY REPERTOIRE AND PEPTIDE CLASSIFIERS THEREFOR

(71) Applicant: ROCHE SEQUENCING SOLUTIONS, INC., Pleasanton, CA (US)

(72) Inventors: Hanying LI, Madison, WI (US); Ken LO, Madison, WI (US); Jigar PATEL, Verona, WI (US)

(21) Appl. No.: 17/287,202

(22) PCT Filed: Oct. 21, 2019
(86) PCT No.: PCT/EP2019/078568

§ 371 Date: Apr. 21, 2021

Related U.S. Application Data
(60) Provisional application No. 62/748,523, filed on Oct. 22, 2018.
(60) Provisional application No. 62/796,711, filed on Jan. 25, 2019.
(60) Provisional application No. 62/887,483, filed on Aug. 15, 2019.

(51) Int. Cl. C12N 15/10; C07K 14/47; G01N 33/564

(52) U.S. Cl. CPC C12N 15/1093; G01N 2800/102; G01N 33/564; C07K 14/4713

(57) ABSTRACT

The present disclosure provides for compositions and methods for identifying peptide classifiers. In one embodiment, a peptide classifier for diagnosing rheumatoid arthritis includes a composition comprising a plurality of molecules. Each molecule comprises a peptide having a sequence selected from SEQ ID NOS: 1-8861, wherein the plurality of molecules defines a classifier for rheumatoid arthritis.

### BACKGROUND

The disclosure relates, in general, to the design and selection of peptides for interrogating biomarkers and, more particularly, to a system and method for identifying and implementing a classifier including one or more variant peptides for diagnostic and predictive applications.

Rheumatoid arthritis (RA) is a progressive autoimmune disease characterized by inflammation and progressive erosion of joint cartilage and bone tissue. Through the seminal works of Schellekens et al. (J Clin Invest. 1998; 101(1):273-81 and Shi et al. (Proc Natl Acad Sci USA. 2011; 108:17372-7), post-translationally modified proteins were isolated from RA synovium and subsequent work demonstrated antibodies against both citrullinated and homocitrullinated proteins to be involved in RA pathogenesis. Development of anti-citrullinated protein antibodies (ACPA) blood tests ensued and is now a part of the American College of Rheumatology (ACR) and European League Against Rheumatism (EULAR) 2010 criteria in RA diagnosis (Arthritis Rheum. 2010; 62: pp. 2569-2581). ACPA positive subsets of RA patients typically follow a more aggressive disease course (Arthritis Res Ther 2005; 7:R949-58), thereby making early diagnosis critical for early intervention to minimize joint erosion and maintain subsequent joint motility for RA patients.

Increasingly, serum profiling using either protein or peptide arrays is becoming commonplace. Notable examples include systemic lupus erythematosus, infectious diseases, and cancer (see Lupus, 27(10), 1670-1678, Theranostics 2017; 7(16):3814-3823, and J Proteome Res. 2017; 16:204-216). While protein arrays may have the advantage of retaining native 3D conformation, complete enzymatic conversion of arginine and lysine to citrulline and homocitrulline, respectively, in recombinant proteins remain a challenge. Peptide arrays, on the other hand, can circumvent these problems by incorporating the necessary monomer during the peptide synthesis process. In the case of peptide arrays synthesized using maskless array synthesis (MAS), photoprotected citrulline and homocitrulline were added to the catalogue of canonical amino acids used during the synthesis process. Substitution of arginine and lysine to citrulline and homocitrulline, respectively, is accomplished during the array design process and incorporation efficiency is limited only by the efficiency of the coupling reaction. Thus, citrulline specific antibody reactivity detected on the peptide array is not conflated by the incomplete conversion of arginine to citrulline in the case of enzymatic conversion.

Antibody binding to its cognate antigen is achieved through non-covalent interactions between the complementarity determining regions (CDR) of the antibody against conformation adopted by the antigen. While structural studies have illustrated the importance of key residues at the interfaces, it is unclear what proportion of the human antibody repertoire recognizes epitopes derived from conformation adopted by contiguous amino acids (linear epitopes) and discontinuous amino acid stretches (conformational epitopes; see PLoS ONE 10:e0121673 and Biomed Res Int 2014, 12), especially in the context of RA. To the inventors' knowledge, an unbiased and comprehensive profiling of serum antibodies in RA serum samples against the entire human proteome, including the citrullinome and the homocitrullinome, has not been reported in the literature.

Accordingly, there is a need for improved processes and systems for the development of new classifiers for both diagnostic and prognostic applications.

### SUMMARY

The present invention overcomes the aforementioned drawbacks by providing a system and method for identification of a classifier for rheumatoid arthritis. An epitope-level characterization of autoantibodies from RA serum samples was performed using a peptide library including both native and citrullinated/homocitrullinated peptides. It is believed that this characterization provides the first unbiased and comprehensive profiling of serum antibodies in RA serum samples against the entire human proteome, including the citrullinome and the homocitrullinome. The results revealed a number of peptide features useful for constructing a classifier for RA, including peptide features that were not previously known to be associated with RA. The present disclosure illustrates how the resulting set of peptide features (SEQ ID NOS: 1-8861) may contribute to the preparation of a plurality of peptide classifiers having actual or predicted properties (i.e., sensitivity and specificity) that meet or exceed those of current commercially available gold-standard tests.

According to one embodiment of the present disclosure, a composition includes a plurality of molecules, each molecule comprising a peptide having a sequence selected from SEQ ID NOS: 1-8861. The plurality of molecules defines a classifier for rheumatoid arthritis.

In one aspect, the classifier discriminates between a sample derived from a first population and a sample derived from a second population. In another aspect, the first population is defined by subjects having at least one marker associated with a first disease state and the second population is defined by subjects lacking the at least one marker associated with the first disease state. In another aspect, the first disease state is rheumatoid arthritis. In another aspect, the marker associated with a first disease state is an antibody. In another aspect the marker associated with a first disease state is a serum marker.

In another aspect, the sample derived from the first population is a serum sample.

In another aspect, the sample derived from the second population is a serum sample.

In another aspect, the classifier discriminates between a sample derived from a first population and a sample derived from a second population. The sample derived from the first population comprises at least one marker associated with a first disease state and the sample derived from the second population lacks the at least one marker associated with the first disease state.

In another aspect, the classifier discriminates between a sample derived from a first population and a sample derived from a second population. At least one marker associated with a first disease state is present in the sample derived from the first population and wherein the at least one marker is absent in the sample derived from the second population.

In another aspect, the first disease state is rheumatoid arthritis.

In another aspect, the marker is one of an anti-citrullinated peptide antibody, and anti-homocitrullinated peptide antibody, an autoantibody, an anti-cyclic citrullinated peptide antibody, and an anti-cyclic homocitrullinated peptide antibody.

In another aspect, the classifier has a specificity of at least 0.90 and a sensitivity of at least 0.70.

In another aspect, the plurality of molecules is configured for binding to the marker associated with the first disease state.

In another aspect, the plurality of molecules comprises at least 3 different molecules.

In another aspect, the plurality of molecules comprises at least 4 different molecules.

In another aspect, the plurality of molecules comprises at least 5 different molecules.

In another aspect, the plurality of molecules comprises at least 6 different molecules.

In another aspect, the classifier has a specificity of at least 0.95 and a sensitivity of at least 0.70.

In another aspect, the classifier has a specificity of at least 0.95 and a sensitivity of at least 0.73.

In another aspect, the classifier has a specificity of at least 0.95 and a sensitivity of at least 0.77.

In another aspect, the classifier has a specificity of at least 0.95 and a sensitivity of at least 0.83.

In another aspect, the classifier has a specificity of at least 0.95 and a sensitivity of at least 0.89.

In another aspect, the classifier has a specificity of at least 0.95 and a sensitivity of at least 0.94.

In another aspect, the classifier distinguishes between a sample derived from a first group defined by a first disease state and a sample derived from a second group defined by a second disease state.

In another aspect, the first group is defined by subjects having a positive diagnosis for rheumatoid arthritis.

In another aspect, the classified distinguishes between the first group and the second group with a sensitivity of at least 0.95 and a specificity of at least 0.77.

In another aspect, the synthetic classifier is one of a diagnostic classifier and a prognostic classifier.

According to another embodiment of the present disclosure, a peptide classifier includes a plurality of molecules, each molecule comprising a peptide having a sequence selected from SEQ ID NOS: 1-8861, the molecules representing at least 4 different sequences selected from SEQ ID NOS: 1-8861.

In another aspect, the plurality of molecules is immobilized on a solid support.

In another aspect, the solid support is one of a microtiter plate, a membrane, a flow cell, a bead, a chip, a slide, a glass surface, and a plastic surface.

According to another embodiment, the present disclosure provides a method for identifying the presence of an antibody indicative of rheumatoid arthritis in a sample. The method includes contacting a sample derived from a subject with a composition according to the present disclosure, binding an antibody present in the sample to at least one of the plurality of molecules, thereby forming an antibody-peptide conjugate, and detecting the antibody-peptide conjugate, thereby identifying the presence of the antibody in the sample.

According to another embodiment, the present disclosure provides a kit for identifying the presence of an antibody indicative of rheumatoid arthritis in a sample. The kit includes a solid support having a plurality of molecules bound thereon, each molecule comprising a peptide having a sequence selected from SEQ ID NOS: 1-8861, and a detectable antibody to a human antibody.

In one aspect, the human antibody is one of an anti-citrullinated peptide antibody, and anti-homocitrullinated peptide antibody, an autoantibody, an and anti-cyclic citrullinated peptide antibody.

According to another embodiment, the present disclosure provides a device for a identifying the presence of an antibody indicative of rheumatoid arthritis in a sample. The device includes a solid support having a plurality of molecules bound thereon, each molecule comprising a peptide having a sequence selected from SEQ ID NOS: 1-8861, the solid support capable of receiving a sample derived from a subject at the location of the plurality of molecules.

In one aspect, the kit further includes at least one of a substrate, a stop solution, a wash buffer, a sample diluent, anti-CCP calibrators, anti-CCP reference controls, and instructions for use. In one aspect, the substrate includes Mg2+, phenolphthalein monophosphate (PMP), and a buffer solution. In another aspect, the stop solution includes sodium hydroxide, EDTA, carbonate buffer (pH>10). In another aspect, the wash buffer includes borate buffer, 0.4% (w/v) sodium azide. In another aspect, the sample diluent includes phosphate buffer, protein stabilizer, 0.5% (w/v) sodium azide. In another aspect, the anti-CCP calibrators include human plasma, buffer, <0.1% (w/v) sodium azide (varying U/mL). In another aspect, the anti-CCP reference control includes human plasma, buffer, <0.1% (w/v) sodium azide (varying U/mL). In another aspect, the positive and negative controls include human plasma, buffer, <0.1% (w/v) sodium azide (varying U/mL).

According to another embodiment, the present disclosure provides a kit for identifying the presence of an antibody indicative of rheumatoid arthritis in a sample. The kit includes a flow cell having a plurality of molecules bound therein, each molecule comprising a peptide having a sequence selected from SEQ ID NOS: 1-8861, the flow cell capable of receiving a sample derived from a subject at the location of the plurality of molecules. The kit further includes a running buffer and instructions for use.

In one aspect, the detectable antibody comprises peroxidase conjugated to an anti-human IgG antibody.

In another aspect, the plurality of molecules comprises peptides having a sequence selected from a first list, the first list consisting of the sequences in Table 1.

In another aspect the plurality of molecules comprises peptides having each of the sequences from the first list.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an example of a method of for identifying a peptide classifier according to the present disclosure.

FIG. 2 is a schematic illustration of 16-mer peptides tiled at either 1 amino acid resolution or 4 amino acid resolution, including a table illustrating tiling of a portion of an example protein sequence Z (X_{A}X_{B}X_{C}X_{D}X_{E}X_{F}X_{G}X_{H}X_{I}X_{J}X_{K}X_{L}X_{M}X_{N}X_{O}X_{P}X_{Q}X_{R}X_{S} . . . ) represented with a series of 10-mer peptides tiled at 1 amino acid resolution, where each Xᵢ represent a single amino acid within the example protein sequence Z.

FIGS. 3A-3F are maps of the RA Abinome for native peptides (FIGS. 3A and 3B), citrullinated peptides (FIGS. 3C and 3D) and homocitrullinated peptides (FIGS. 3E and 3F) for RA samples (FIGS. 3A, 3C, and 3E) and control samples (FIGS. 3B, 3D, and 3F). Peptides are organized by their chromosomal locations and the height of the bar indicates the proportion of samples that contains significant antibody reactivity as detected on the peptide array.

FIG. 4 is a heatmap of log-transformed array signals derived from the array. Peptides are organized in rows and serum samples organized in columns. Sample condition is indicated under the dendrogram (black—control and white—RA) and CCP2 status is overlaid onto the sample condition (+/−indicate CCP2 status). Heatmaps are trellized also by native peptides (Native)—left, citrullinated peptides (Citrullinated)—middle, and homocitrullinated peptides (Homocitrullinated)—right.

FIGS. 5A and 5B shows a side-by-side comparison between fluorescence signal derived from array (FIG. 5A) and the signal histogram obtained using a nanoliter-scale immunoassay system (FIG. 5B) for 8 representative serum samples (4 controls and 4 RA) for protein PATE4. In FIG. 5A, signal derived from native peptides (Native) is shown in the first and third columns, whereas signal derived from citrullinated peptides (Citrullinated) is shown in the second and fourth columns. Individual serum samples are organized in rows, with the left two columns representing control serum sample data and the right two columns representing RA serum samples data. The epitope that went onto validation studies is indicated by shading centered at position 30, and had the sequence CNTCIYTEGWKCMAG-R/cit-GTCIAKENELCS (SEQ ID NO: 1), where R/cit indicates the use of either arginine (R) or citrulline (cit) at the indicated position. FIG. 5B shows signal histograms generated using the nanoliter-scale immunoassay system organized in the same way as that of array signal. With reference to the x-y-z coordinate system (bottom left), the x-axis indicates radius in μm (ranging from 0 to 900), the y-axis indicates angle in μm (ranging from 100 to 600), and the z-axis indicates intensity in relative units (ranging from 0 to 1).

FIGS. 6A and 6B illustrate the performance of an 8-epitope RA diagnostic classifier on the nanoliter-scale immunoassay system (see also Table 1). Epitopes (V1, V3, V5, V17, V19, V27, V28 and V29) are organized in rows and samples in columns. Filled squares indicate positivity and empty squares indicate negativity for the specific epitope. Sample conditions are indicated above the plots (control vs. RA). CCP2 status of the serum samples are indicated by +/−. FIG. 6A shows the initial expanded cohort of 92 patients (29 controls and 63 RA). FIG. 6B shows the independent validation cohort of 181 patients (54 controls and 127 RA).

FIG. 7 is a plot illustrating the proportion of probes with significant reactivity by condition (CCP2 negative [−] RA, CCP2 positive [+] RA, and control) organized by substitution categories (i.e., native, citrullinated and homocitrullinated). Each point represents a specific sample.

### DETAILED DESCRIPTION

### I. Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Abinome: As used herein, the term “Abinome” refers to antibody repertoire observed against the annotated proteome or antibody reactome.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level, and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises or consists of an organism, such as an animal or human. In some embodiments, a biological sample comprises or consists of biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; other body fluids, secretions, and/or excretions; and/or cells therefrom, etc. In some embodiments, a biological sample comprises or consists of cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Citrullinated: As used herein, the term “Citrullinated” refers to a peptide or protein in which each arginine residue is replaced with a citrulline residue.

Citrullinome: As used herein, the term “Citrullinome” refers to antibody repertoire observed against citrullinated peptides from the annotated proteome, where arginine is substituted to citrulline.

Classifier: As used herein, the term “Classifier” refers to a tool for distinguishing the identity of one or more samples and assigning the identified samples to one or more categories. More generally, a classifier is useful for identifying to which of a set of categories (sub-populations) a new observation belongs, on the basis of a training set of data containing observations (or instances) whose category membership is known. Accordingly, a classifier can encompass predictive models, algorithms, charts, look-up tables, etc., that enable a new observation to be categorized. A classifier can assign observations to binary categories such as yes/no, healthy/diseased, present/absent, and the like. Alternatively (or in addition), a classifier can assign observations to a broader set of categories, such as a set of different types [type 1, type 2, type 3, . . . , type i], a set of different disease states [healthy, disease A, disease B, . . . , disease Z], a set of response predictions for different treatment options [non-responder, option A responder, option B responder, . . . , option Z responder], a set of treatment options [no treatment, treatment A, treatment B, . . . , treatment Z], the like, and combinations thereof.

Comprising: A composition or method described herein as “comprising” one or more named elements or steps is open-ended, meaning that the named elements or steps are essential, but other elements or steps may be added within the scope of the composition or method. It is to be understood that composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Those of ordinary skill in the art, reading the present specification, will appreciate that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Feature: As used herein, the term “Feature” refers to an element of a predictive model (e.g., a classifier) that distinguishes the identity of one or more samples and assigns the samples into one or more categories. The term “feature” may be used interchangeably with the term “predictor” in the context of a statistical model.

Homocitrullinated: As used herein, the term “Homocitrullinated” refers to a peptide or protein in which each lysine residue is replaced with a homocitrulline residue.

Homocitrullinome: As used herein, the term “Homocitrullinome” refers to antibody repertoire observed against homocitrullinated peptides from the annotated proteome, where lysine is substituted to homocitrulline.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Sample: As used herein, the term “sample” refers to a substance that is or contains a composition of interest for qualitative and or quantitative assessment. In some embodiments, a sample is a biological sample (i.e., comes from a living thing (e.g., cell or organism). In some embodiments, a sample is from a geological, aquatic, astronomical, or agricultural source. In some embodiments, a source of interest comprises or consists of an organism, such as an animal or human. In some embodiments, a sample for forensic analysis is or comprises biological tissue, biological fluid, organic or non-organic matter such as, e.g., clothing, dirt, plastic, water. In some embodiments, an agricultural sample, comprises or consists of organic matter such as leaves, petals, bark, wood, seeds, plants, fruit, etc.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the word “synthetic” means produced by the hand of man, and therefore in a form that does not exist in nature, either because it has a structure that does not exist in nature, or because it is either associated with one or more other components, with which it is not associated in nature, or not associated with one or more other components with which it is associated in nature.

Synthetic Peptide: As used herein, the term “synthetic peptide” refers to a peptide that differs from a naturally occurring peptide at one or more amino acid positions. In one aspect, a synthetic peptide can be differentiated from both a wild-type peptide and a mutant or other naturally occurring peptide. For example, a wild-type peptide can consist of a peptide sequence defining at least a portion of a wild-type protein sequence. In some cases, the wild-type protein may be known to occur naturally in a mutant form. For example, in certain autoimmune diseases, selected proteins are observed to include one or more citrulline residues in place of arginine residues. Notably, this citrullination is known to occur in nature. In this case, an example mutant peptide can consist of a peptide sequence defining at least a portion of the citrullinated protein sequence. However, the mutant peptide including the one or more citrulline residues can still be considered to be a naturally occurring peptide. By contrast, a synthetic peptide will differ from a wild-type peptide, a mutant peptide, or another naturally occurring peptide sequence defining at least a portion of a naturally occurring protein sequence. In one example, a synthetic peptide can include one or more amino acid substitutions, deletions, insertions, other like modifications, or a combination thereof, where the aforementioned modifications are not observed in a naturally occurring form of the protein sequence to which the peptide corresponds. For example, a synthetic peptide can include one or more citrulline residues in place of arginine residues, where the citrullinated arginine is not observer to occur in nature, either as a wild-type or mutant peptide.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively, or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a variant may also have one or more functional defects and/or may otherwise be considered a “mutant”. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### II. Detailed Description of Certain Embodiments

As also discussed above, in various situations it may be useful to provide a method for accurately diagnosing a subject for a particular condition, disease, or the like. Depending on the nature of the diagnostic, the method or test can enable early detection, potentially resulting in improved opportunities to plan for treatment and the like. However, a given affliction may be difficult to accurately diagnose, especially early on, when detectable symptoms are restricted to changes at the molecular level (e.g., genomic mutations, protein aggregation, changes in expression levels of nucleic acids or proteins, and the like) that may not have manifested in more readily detectable ways. For example, Alzheimer's disease (AD) is a chronic neurodegenerative disease that can include such outward manifestations as memory loss, confusion, and decreased or poor judgment. However, while the cause of AD is poorly understood, biochemical changes including protein misfolding are hypothesized to contribute to the progression of the disease. Accordingly, examination of brain tissue is presently needed for a more definite diagnosis. That is, diagnosis of a disease, such as AD, may benefit from a diagnostic that focuses on features such as peptides, proteins, or nucleic acids as opposed to behavioral characteristics or other outward manifestations that can be subjective or difficult to detect early in the progression of the condition.

In another example, it may be useful to provide a method for accurately forecasting the probable course of a disease or determining whether a subject may respond to a given course of treatment (i.e., a prognostic or predictive method). More than one treatment method is often available for use; however, if no predictive test is available to indicate which treatment or treatments will be effective, it may be necessary to rely on trial and error, attempting multiple different treatments either alone or in combination to determine which treatments will be effective. Ultimately, in the absence of either or both of a diagnostic and prognostic method or test, several challenges may arise in the diagnosis and treatment of a subject.

These and other challenges may be overcome with a system and method for the design and implementation of a peptide classifier. In one aspect, a classifier can be implemented to solve the problem of categorizing a subject within a population of subjects. For example, a classifier may assign a subject (or observation about that subject) to a particular category or sub-population based on a training set of data containing information about one or more different subjects (or observations) within the population. An example would be assigning a diagnosis to a given subject as determined by observed characteristics of the subject.

The present disclosure is, at least in part, based on the surprising discovery that a set of one or more peptides, including non-naturally occurring variants of known peptide sequences can be used to prepare one or both of a diagnostic classifier and a prognostic classifier for categorizing an observation or aspect of a group of subjects. For example, an observation about the interaction of the peptides with a serum sample collected from the subject can be used to diagnose the subject for a given condition, predict which treatment or treatments may be effective for the subject, the like, and combinations thereof.

In applying the above approach, the inventors have discovered a plurality of peptide features that are useful for identifying biomarkers associated with RA, including a number of peptides features that have not previously been linked to RA. Accordingly, the present disclosure provides for novel classifiers useful for diagnosing RA. Moreover, the present disclosure provides for general methods of preparing classifiers useful for diagnosing RA from the disclosed list of peptide features (SEQ ID NOS: 1-8861).

In one aspect, a peptide classifier according to the present disclosure can include wild-type, mutant, or synthetic peptides that are differentiable from a traditional classifier for querying a biomarker. A biomarker can be defined as a naturally occurring, biological element (e.g., a nucleic acid, a protein, a small molecule, an antibody, or the like) that can be detected in the blood, serum, urine, or another fluid of a subject. In one aspect, the biomarker may be produced by a foreign (non-native) or mutant (native) element in the subject (e.g., a tumor, a virus, a parasite, or the like) or in response to the presence of the native or non-native element. In general, querying of biomarkers can allow for early detection of a condition, confirmation of a diagnosis, predicting an outcome or making a prognosis, monitoring treatment response, and the like. Whereas some classifiers can include one or more elements for querying biomarkers such as normal or mutant peptides or proteins, the synthetic peptides of the present disclosure cannot be properly equated to these normal or mutant peptides or proteins. In one aspect, the synthetic peptides of the present disclosure may be variants of normal (wild-type) or mutant versions of peptides or proteins that may exist in a given subject or may be associated with a given condition. While the native peptides (wild-type or mutant) may be predicted to be useful for querying biomarkers as they may be observed to interact with those biomarkers in nature, the synthetic peptides of the present disclosure are non-naturally occurring, designed sequences that are absent in the curated proteome. However, these synthetic peptides may contribute to a more sensitive and/or specific classifier for querying those same biomarkers. Without being limited by any particular theory, it is hypothesized that the synthetic peptides of the present disclosure may adopt a conformation that it is better suited to interact with or be bound by a portion of a serum antibody or another biomarker relative to naturally occurring peptide sequences. Importantly, the synthetic peptides of the present disclosure can be capable of detecting or otherwise interacting with one or more biomarkers derived from a subject as the basis of a diagnostic or prognostic/predictive synthetic classifier.

In one aspect, the present disclosure leverages the surprising discovery that a synthetic or variant peptide sequence can be used to provide a classifier, as one would not necessarily expect to find non-naturally occurring peptides that can be used as a classifier to discriminate between naturally occurring biomarkers. In light of this result, the present disclosure provides for systems and methods to design peptide-based probes for detection of biomarkers that may be useful in one or more of predictive, prognostic, diagnostic, pharmacodynamic, and/or efficacy-response applications. As further defined herein, a biomarker is a measurable substance in an organism whose presence is indicative of some phenomenon such as disease, infection, or environmental exposure. Methods according to the present disclosure for detection of one or more biomarkers include i) systematic screening of known peptide targets, and ii) derivatization of the aforementioned peptides, including systematic mutation of known sequences with both natural and non-natural amino acids, cyclization of peptides and their mutant counterparts, or a combination thereof to provide a plurality of synthetic variant peptides. Derivatization is based on the ability to distinguish between sub-groups of biomarker populations (e.g., drug responders vs. non-responders, or diseased vs. control/healthy populations) in a disease area.

In one aspect, the present disclosure overcomes the challenge of having to rely solely on screening to identify peptide candidates and using them as probes to query biomarkers. Existing solutions rely on methods such as phage or mRNA display for natural amino acids substitution. As for non-natural amino acids such as citrulline and homocitrulline, work is ongoing to overcome the challenge of incorporating non-natural amino acids into various display technologies (e.g., mRNA display, phage display, etc.) via genetic code expansion or genetic code reprogramming. By contrast, embodiments of the present disclosure involve systematically mutating these peptide candidates to find variant peptide sequences (i.e., synthetic peptides) that perform better than the original, naturally occurring candidate peptides as probes for querying biomarkers. These variant peptide sequences are unlikely to be found in the human proteome (natural vs. non-natural), and are at least unknown (i.e., non-naturally occurring) variants of the portions of the proteins from which they are derived. In one aspect, the peptides of the present disclosure can be implemented in detection schemes for the accurate diagnosis of a subject with a given condition as well as for informing which treatments may be effective for a given subject.

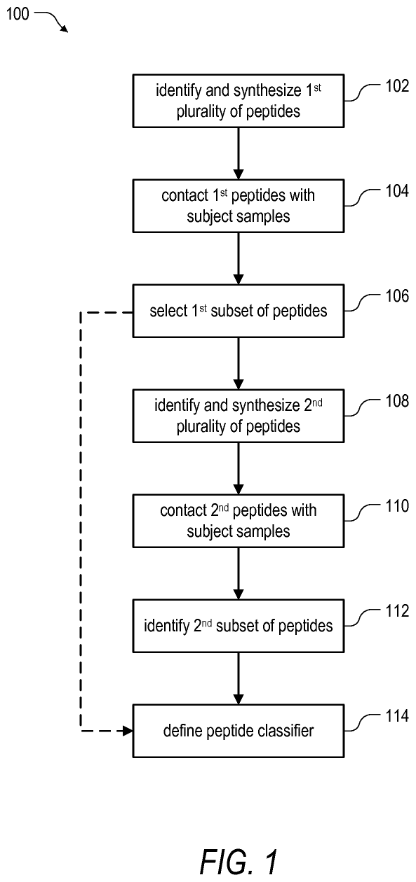

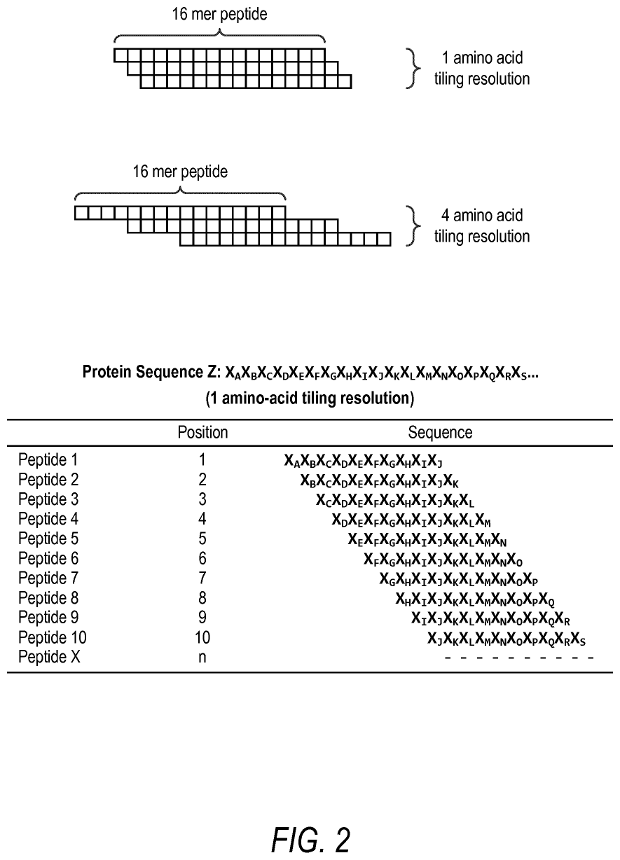

Turning to FIG. 1, an embodiment of a method 100 for identifying a peptide classifier includes a step 102 of identifying and synthesizing a first plurality of peptides. At least a portion of the first plurality of peptides can define at least one naturally occurring amino acid sequence. For example, the peptides can be tiled at a given amino acid resolution (see FIG. 2) along the length of an entire partial or full-length protein sequence of interest. In some embodiments, the peptides can have amino acid sequences that collectively represent the entire human proteome or another proteome of interest. Moreover, the peptides can have amino acid sequences that collectively represent a modified or variant version of at least a portion of the entire human proteome or another proteome of interest. For example, the peptides can be partially or completely citrullinated and/or homocitrullinated relative to the native or wild-type sequence.

A next step 104 of the method 100 includes contacting at least a first sample and a second sample with a first plurality of peptides. The first sample is derived from a first group of a cohort, and the second sample derived from a second group of the cohort. The first group is different from the second group. The cohort is generally a group of subjects with a common defining characteristic. For example, the cohort can be a group of subjects, where a portion of the subjects have been diagnosed with (or suspected of) a particular condition or disease. More particularly, the first group within the cohort can be a group of healthy (control) subjects and the second group within the cohort can be a group of subjects known or suspected to have a particular condition, disease, diagnosis, or the like.

Notably, samples can be derived from two or more groups within a cohort. As described in the Examples below, in some embodiments, a cohort can include three or more groups. Example groups can include at least a control or healthy subject group, a group of subjects diagnosed with a particular condition where the subjects responded to a particular treatment, and a group of subjects diagnosed with the same particular condition where the subjects did not respond to the particular treatment. Example groups can further include a first group of subjects diagnosed with a first condition and a second group of subjects diagnosed with a second condition different from the first condition. In one aspect, the first and second conditions may be related, such as different types of arthritis (e.g., RA and osteoarthritis) or different types of cancer. Accordingly, the method 100 can be used to identify a peptide classifier for distinguishing between samples associated with the first condition and the second condition. Moreover, more than a single sample may be tested from each of the groups within the cohort. For example, at least 2, 3, 4, 5, 6, 7, 8, 9, 10, 15, 20, 30, 50, 100, 1,000, 10,000, or more samples can be obtained for each group within a cohort. Each of the samples from a selected group of the cohort can be contacted individually or in combination with one or more peptides as described below. In one aspect, a sample can be a blood sample, a serum sample, a buccal swab, a urine sample, a stool sample, a tissue sample, the like, or combinations thereof. The single sample will generally be collected from an individual subject. However, in some embodiments, it may be useful to pool one or more samples to provide a single, combined sample.

A next step 106 of the method 100 includes selecting a first subset of peptides from the first plurality of peptides. The first subset of peptides can be candidate peptides that can at least partially classify the first and second samples from the first and second groups. Having identified a plurality of peptides in the step 106, the method 100 can optionally proceed to the step 114 of defining a peptide classifier with the identified peptides from the first plurality of peptides. Alternatively, or in addition, a next step 108 of the method 100 includes identifying and synthesizing a second plurality of peptides. At least a portion of the second plurality of peptides can define the sequences of the first subset of peptides and a plurality of variant peptides of the first subset of peptides. For example, the plurality of variant peptides can include, for each one of the first subset of peptides, a variant peptide having at least one of a substitution, a deletion, an insertion, an extension, and a modification. In one aspect, the plurality of variant peptides can include one or more synthetic peptides as defined herein.

A next step 110 of the method 100 can include contacting at least the first sample and the second sample (or samples comparable thereto) with a second plurality of peptides. Thereafter, a next step 112 of the method 100 can include identifying or otherwise selecting a second subset of peptides from the second plurality of peptides. The second subset of peptides can include at least one of the plurality of variant peptides. In a next step 114 of the method 100, a peptide classifier can be defined including at least one of the second subset of peptides. The peptide classifier can distinguish between a sample derived from the first group and a sample derived from the second group. Moreover, the peptide classifier can include one or more synthetic peptides identified according to the method 100, thereby defining a synthetic classifier.

Notably, the embodiments of the method 100 according to the present disclosure can include one or more additional steps or omit one or more of the illustrated steps of the method 100. For example, it may be possible to identify a subset of peptides from which a plurality of variant peptides can be prepared without performing the initial steps of the method 100. That is, it may be possible to omit the steps of identifying and synthesizing a first plurality of peptides, contacting the first plurality of peptides with the subject samples, and selecting the first subset of peptides based on the outcome of the first and second illustrated steps of the method 100 of identifying and contacting. Accordingly, the method 100 can begin with a step of identifying and synthesizing the second plurality of peptides. In general, the method 100 can be modified in any suitable way that still enables the outcome of defining a classifier, whether the resulting classifier is synthetic or otherwise. Yet other variations of the method 100 that fall within the scope of the present disclosure will be apparent from the additional examples and description included herein.

The inventors have discovered that aspects of the present disclosure can be applied to the identification of a peptide classifier for RA. Autoantibodies against citrullinated proteins are found in 64-89% of RA patients, with a specificity of 88-99%. While citrullinated vimentin, fibrin and histone have been implicated as targets of autoantibody reactivity, new targets, such as Tenascin-C, continue to be uncovered. To this end, an epitope-level characterization of autoantibodies from RA serum samples was performed using a peptide library including both native and modified peptides. In particular, the present disclosure provides, in part, for the first unbiased and comprehensive profiling of serum antibodies in RA serum samples against the entire human proteome, including the citrullinome and the homocitrullinome.

In identifying a peptide classifier for RA, peptide libraries were prepared comprising over 4.6M peptides representing the entire annotated human proteome from the UniProt database. A total of 20,246 proteins were represented as overlapping 16-mer peptides with a four amino acid tiling resolution (see FIG. 2). In addition to native peptides for autoantibody profiling, citrullinated and homocitrullinated peptides were also included on the array, substituting for each arginine and each lysine, respectively, providing a comprehensive screen against all possible epitopes. Notably, the peptide library also included peptides having a combination of citrullinated and non-citrullinated arginine positions, for example, in the case that a peptide sequences included two or more arginine positions. Immunoglobulin G (IgG) antibodies for 26 serum samples (8 controls and 18 RA samples) were profiled. Comprehensive antibody profiling resulted in the surprising discovery of many citrullinated peptides and proteins that have not been previously reported to be associated with RA.

Using information derived from peptide array studies, an 8-epitope RA diagnostic classifier was constructed and subsequently validated using a nanoliter-scale immunoassay system (GYROLAB XPLORE). A cohort of 92 samples (29 controls and 63 RA samples) was then evaluated on the nanoliter-scale immunoassay system with the RA diagnostic classifier obtaining a 96% specificity and a 92% sensitivity in performance. The classifier was further validated with an independent cohort of 181 serum samples (54 controls and 127 RA samples), yielding a 95% specificity and an 85% sensitivity in performance.

The present disclosure is believed to provide the first unbiased proteome level antibody profile for RA, which is defined herein as the “RA Abinome”. As has been previously characterized, antibodies against citrullinated epitopes are readily seen in RA serum samples and ACPA positive RA patients. The disclosed data shows how extensive antibody reactivity against the citrullinated proteome is and seems to occur throughout the proteome. Relative to ACPA reactivity, antibody reactivity against homocitrullinated epitopes (anti-CarP) was seen less frequently in this cohort, which is consistent with seminal reports by Shi et al. (Proc Natl Acad Sci USA. 2011; 108:17372-7). Interestingly, while anti-CarP antibody was observed in 4 out of 18 RA serum samples tested, these individuals also showed ACPA reactivity and did not constitute an anti-CarP positive and ACPA negative subgroup of RA patients. This is also consistent with Peccani et al. (Arthritis Res Ther. 2016; 18:276), where the majority of anti-CarP positive patients were also ACPA positive.

Gene Ontology (GO) term enrichment analysis was performed for significant reactivity for both RA and control groups. For autoantibody reactivity, there was no specific GO term enrichment unique to RA group over control (Table 1). For anti-CarP reactivity, only one molecular function GO term “actin filament binding” was found to be enriched. ACPA reactivity, on the other hand, had 33 biological processes, 50 cellular components and 51 molecular function GO terms that were enriched at a Benjamini-Hochberg corrected p-value of 0.05. While 50 cellular components GO terms were enriched, most top statistical hits were intracellular such cytoplasm and cytosol. This, coupled with the fact that no observable preference for extracellular peptides was seen in the RA Abinome by student t-test (p-value=0.75) is consistent with the idea that necrotic tissue is a significant source of antigens for citrullination by peptidyl arginine deaminases (PADs) within the RA synovium (Arthritis Res Ther. 2016; 18:239).

TABLE 1

| Epi- | UniProt | Protein | |
|---|---|---|---|
| tope | ID | Name | Peptide Sequence (N′ → C′) |
| V1 | P0C8F1 | PATE4 | CNTCIYTEGWKCMAG-Cit/R- |
| | | | GTCIAKENELCS (SEQ ID NO: 1) |
| V3 | Q9UMX5 | NENF | KGVVFDVTSGKEFYG-Cit/R- |
| | | | GAPYNALTGKDS (SEQ ID NO: 2) |
| V5 | Q9UMX5 | NENF | YKAKYPIVGYTA-Cit/R-Cit/R- |
| | | | ILNEDGSPNLDFKP (SEQ ID NO: 3) |
| V17 | Q8N967 | LRTM2 | MEWFSY-Cit/R-GG-Cit/R- |
| | | | LDQLACTLP (SEQ ID NO: 4) |
| V19 | O96014 | WNT11 | -hc/K-NE-hc/K-VGSHGTQDQCN- |
| | | | hc/K-TSNGSDSCDLM (SEQ ID NO: |
| | | | 5) |
| V27 | Q5T848 | GP158 | IYGLQPNLVPEF-Cit/R- |
| | | | GVMKVDINLQKVDID (SEQ ID NO: |
| | | | 6) |
| V28 | Q5H8C1 | FREM1 | VLEVPEFNGLSQAID-hc/K-NLLR |
| | | | (SEQ ID NO: 7) |
| V29 | O75443 | TECTA | DCPN-Cit/R-TCELGNG-Cit/R- |
| | | | ELCGCIE (SEQ ID NO: 8) |

Cit/R indicates peptide sequence positions where distinct peptides were prepared for each of citrulline (Cit) and arginine (R), while hc/K indicates peptide sequence positions where distinct peptides were prepared for each of homocitrulline (hc) and lysine (K).

Overall signal between the peptide array libraries and the nanoliter-scale immunoassay system were comparable even though differences were expected due to differences in surface chemistry between the two platforms. Peptide array signal exhibited higher dynamic range, as saturation effects were observed in the nanoliter-scale immunoassay system with epitopes with strong peptide array signal, coupled with regression coefficients being less than 1 in all epitopes used in validation. When comparing the signal log ratios of citrullinated and homocitrullinated peptides versus native peptides between the two platforms, regression coefficients of determination varied from 0.32 to 0.78, depending on the epitope.

Translating findings between immunoassays can be challenging as surface characteristics can impact the conformation of the epitopes and subsequent presentation and binding of antibodies against the cognate partners (J. R. Soc., Interface 2012, 9, 2688-2695). According the present disclosure, array signal was treated qualitatively in order to overcome the aforementioned challenge. When comparing qualitative calls between the two platforms, the overall concordance rate was 86.4% when all data was combined.

Whole proteome screening of linear epitopes has one specific shortcoming, namely, there is no assurance that short linear peptides can adopt native protein confirmation. The inventors have previously shown that linear peptides can adopt the necessary conformation needed for protein binding to occur (Scientific Reports. 7. 10.1038/s41598-017-12440-1). While there is much debate as to the relative proportion of human antibodies binding linear epitopes versus conformational/discontinuous epitopes (PLoS ONE 10:e0121673; Biomed Res Int 2014, 12), the present disclosure provides for a comprehensive, whole proteome screen of linear epitopes can be potentially informative for RA diagnosis. By mining the RA Abinome data from peptide arrays, candidate epitopes were screened using the nanoliter-scale immunoassay system with traditionally synthesized peptides to avoid potentially spurious findings that are peptide array specific. By combining eight epitopes, the overall diagnostic performance of the presently disclosed classifier was 95% specific and 92% sensitive on our initial 92-sample cohort. To provide context as to the percentage of RA samples that are ACPA positive, the CCP2 assay was performed on the 92-sample cohort. Overall, the tested RA samples were positive by CCP2 assay 68.3% of the time, while our controls were positive 3.4% of the time, yielding 96.6% specificity and 68.3% sensitivity. Notably, the 8-epitope diagnostic classifier according to the present disclosure outperforms the current gold standard for RA diagnostic blood test (i.e., the CCP2 assay) in terms of sensitivity while being comparable in terms of specificity. Finally, in order to assess the robustness of the presently disclosed 8-epitope classifier, the diagnostic performance was further validated with an independent, commercially purchased cohort of 181 patients. The validation performance was 95% specific and 85% sensitive.

As demonstrated by the presently disclosed 8-epitope diagnostic marker, it is evident that whole proteome profiling of linear epitopes has potential utility in revealing novel insights for disease prognosis and diagnosis of RA. It will therefore be appreciated that the aforementioned 8 epitope classifier (Table 1) is only one of many possible peptide classifiers that can be constructed from the peptide features identified with the peptide libraries described herein. In particular, Table 2 shows the number of different feature sets that can be selected from SEQ ID NOS: 1-8861 to define a classifier for RA as predicted by a machine learning algorithm. The algorithm begins with a seed feature and iteratively searches for features that maximally increases sensitivity while minimizing decreases in specificity in a greedy manner. The algorithm stops when either (a) sensitivity is perfect, (b) no additional features are found to be useful or (c) maximum number of features is reached. All classifiers that pass the specified sensitivity and specificity thresholds are returned to the user. The number of features used to construct each classifier ranged from 1 to 6, the sensitivity of each classifier ranged from 0.78 to 1.00, and the specificity of each classifier was constant at 1.00. The remaining columns of Table 2 list the resulting number of classifiers identified at each sensitivity/specificity combination using the machine learning algorithm for increasing numbers of unique features.

TABLE 2

| | | Number of Unique Features in Classifier | | | | | |
|---|---|---|---|---|---|---|---|
| Sensitivity | Specificity | 1 | 2 | 3 | 4 | 5 | 6 |
| 0.78 | 1.00 | 1 | 71 | 0 | 0 | 0 | 0 |
| 0.83 | 1.00 | 0 | 150 | 31 | 0 | 0 | 0 |
| 0.89 | 1.00 | 0 | 18 | 167 | 31 | 0 | 0 |
| 0.94 | 1.00 | 0 | 0 | 38 | 167 | 31 | 0 |
| 1.00 | 1.00 | 0 | 0 | 0 | 38 | 167 | 31 |

By way of example, for groups of 3 unique features selected from SEQ ID NOS: 1-8861 by the machine learning algorithm, no classifiers were constructed with a sensitivity of 0.78, 31 classifiers were constructed with a sensitivity of 0.83, 167 classifiers were constructed with a sensitivity of 0.89, 38 classifiers were constructed with a sensitivity of 0.94, and no classifiers were constructed with a sensitivity of 1.0. As stated above, each predicted 3-feature classifier had a specificity of 1.00. Notably, the machine learning algorithm identified a total of 236 different classifiers consisting of 6 or fewer unique features that had a predicted sensitivity and specificity of 1.00. Although not shown in Table 2, it will be appreciated that further classifiers including 7 or more features could be identified by the machine learning algorithm with a predicted sensitivity and specificity of 1.00.

### EXAMPLES

The following Examples are meant to be illustrative and are not intended to be limiting in any way.

### Example 1

To identify a peptide classifier for RA, whole proteome peptide libraries were designed using an array-format based on 20,246 protein sequences obtained from the Universal Protein Resource (UniProt) downloaded on Apr. 12, 2013 (Query: taxonomy: “Homo sapiens (Human) [9606]” keyword: “Complete proteome [KW-0181]” AND reviewed:yes). The sequences were tiled as overlapping 16-mer linear peptides at a four amino acid tiling interval (see FIG. 2). A peptide array design was then prepared by randomly distributing the resulting 16-mer linear peptide sequences across the surface of the array. In addition to native peptides for autoantibody profiling, citrullinated and homocitrullinated peptides were also included on the array, substituting for arginine and lysine, respectively. The total number of peptide probes present in the whole proteome array design was 2,014,531 native peptides, 1,363,951 at least partially citrullinated peptides, and 1,300,186 at least partially homocitrullinated peptides, for a total of 4,678,668 peptide probes.

Microarrays were synthesized with a MAS by light-directed solid-phase peptide synthesis using an amino-functionalized plastic support coupled with a 6-aminohexanoic acid linker and amino acid derivatives carrying a photosensitive 2-(2-nitrophenyl) propyloxycarbonyl (NPPOC) protection group. Amino acids (final concentration 20 mM) were pre-mixed for 5 min in N,N-Dimethylformamide (DMF) with N,N,N′,N′-Tetramethyl-O-(1H-benzotriazol-1-yl)uranium-hexafluorophosphate (HBTU; final concentration 20 mM) as an activator, 6-Chloro-1-hydroxybenzotriazole (6-Cl-HOBt; final concentration 20 mM) to suppress racemization, and N,N-Diisopropylethylamine (DIPEA; final concentration 31 mM) as base. Activated amino acids were then coupled to the array surface for 3 min. Following each coupling step, the microarray was washed with N-methyl-2-pyrrolidone (NMP), and site-specific cleavage of the NPPOC protection group was accomplished by irradiation of an image created by a Digital Micro-Mirror Device (HD 1080p resolution), projecting 365 nm wavelength light. Coupling cycles were repeated to synthesize the full in silico-generated peptide library. Prior to sample binding, final removal of side-chain protecting groups was performed in 95% trifluoroacetic acid (TFA), 0.5% Triispropylsilane (TIPS) for 30 min. Peptide array synthesis using a MAS is further described in U.S. Pat. No. 10,161,938 by Patel et al.

For processing and analysis of peptide arrays, arrays were incubated twice in methanol for 30 s and rinsed four times with reagent-grade water. Arrays were washed for 1 min in TBST (1×TBS, 0.05% Tween-20), washed twice for 1 min in TBS, and exposed to a final wash for 30 s in reagent-grade water. Slides were then spun dry in a microarray dryer.

Samples were diluted 1:100 in binding buffer (0.01M Tris-Cl, pH 7.4, 1% alkali-soluble casein, 0.05% Tween-20) and bound to arrays overnight at 4° C. After sample binding, the arrays were washed three times in wash buffer (1×TBS, 0.05% Tween-20), 10 min per wash. Primary sample binding was detected via ALEXA FLUOR 647-conjugated goat anti-human IgG secondary antibody diluted to 1:10,000 (final concentration 0.1 ng/μl) in secondary binding buffer (1×TBS, 1% alkali-soluble casein, 0.05% Tween-20). Arrays were incubated with secondary antibody for 3 h at room temperature and then washed three times in wash buffer (10 min per wash), 30 see in reagent-grade water and spun dry. Fluorescent signal of the secondary antibody was detected by scanning at 635 nm at 2 μm resolution and 15% PMT gain, using a microarray scanner.

Array data analyses were performed in the R statistical programming environment version 3.2.3 using a custom developed R package. Raw array signal intensities were spatially corrected via a 2-D loess smoother (Statistical Models in S, 2017, edited by T. J. Hastie) and background corrected by deconvolution (Bolstad 2004. Low Level Analysis of High-Density Oligonucleotide Array Data: Background, Normalization and Summarization). A one-sided Kolmogorov-Smirnov test was used to assess whether the signal within an 8-mer sliding window centered on a specific probe is above sampled background (Methods in Enzymology, 411, 270-282). A signal intensity exceeding 2¹² (or 4096 fluorescence units) with a sliding window significance of 0.05 was used to categorize significant antibody reactivity. An epitope is defined as two or more contiguous (including overlapping) probes with significant reactivity.

Hierarchical clustering was performed on log transformed array signal intensities using peptide probe intensities belonging to significant epitopes in 3 or more samples within the 26 serum samples profiled on the whole proteome array, using the R package ‘hclust’. Hierarchical clustering was performed separately for citrullinated, homocitrullinated, and native peptide probes.

Proteins with at least one significant epitope in either citrullinated, homocitrullinated, or native peptide probes were submitted for GO enrichment analysis via the DAVID Bioinformatics Resource 6.8. Enrichment of specific GO terms in ‘molecular functions’, ‘subcellular component’ and ‘binding proteins’ was done via a hypergeometric test against all annotated human proteins. P-values were corrected using Benjamini-Hochberg and GO terms with P values less than 0.05 are listed in supplemental data (Nature Protoc. 2009; 4(1):44-57; Nucleic Acids Res. 2009; 37(1):1-13). All data graphics were made using the R package “ggplot2” and any additional statistical analyses were conducted in R.

For further analysis of peptides with the nanoliter-scale immunoassay system, peptides were prepared using traditional solid phases synthesis with Fmoc and Boc chemistry and a solid support resin. Peptides were further synthesized with C-terminal amidation and biotinylation via the side chain of the C-terminal lysine. Peptides were then assayed using the nanoliter-scale immunoassay platform (GYROLAB XPLORE). The platform automates finely controlled immunoassays in identical microfluidic channels using highly sensitive, laser-induced fluorescence (LIF) detection. The 3-step assay was carried out as per manufacturer's instructions. During the Capture phase of the 3-step assay, 1 μM of peptide in 1× phosphate buffered saline+Tween (PBST) was used. Serum samples were diluted to 10% in 1× HNmax buffer. The ALEXA FLUOR 647 conjugated goat anti-human Fc secondary antibody (1 mg/mL stock) was diluted to 3.6 μg/mL in 1× Rexxip F buffer. Quantification of signal intensity was obtained by GYROLAB EVALUATOR software using signal at the 5% PMT scan setting.

After signal extraction, signal specific to each citrullinated or homocitrullinated peptide was converted to a ratio against the corresponding native peptide, which was termed “signal-to-noise” ratio. The signal from a no peptide control was used for peptide probes with signal not specific to citrullinated, homocitrullinated, or native peptides.

To carry out CCP2 assays, a CCP2 IgG ELISA Kit was purchased from a commercial manufacturer (ABNOVA). The CCP2 assay was conducted as per the manufacturer's instructions with the exception that after adding chromogenic substrate solution, the reaction was stopped after 5 min. The resulting colorimetric intensity was measured at 450 nm using a microplate reader.

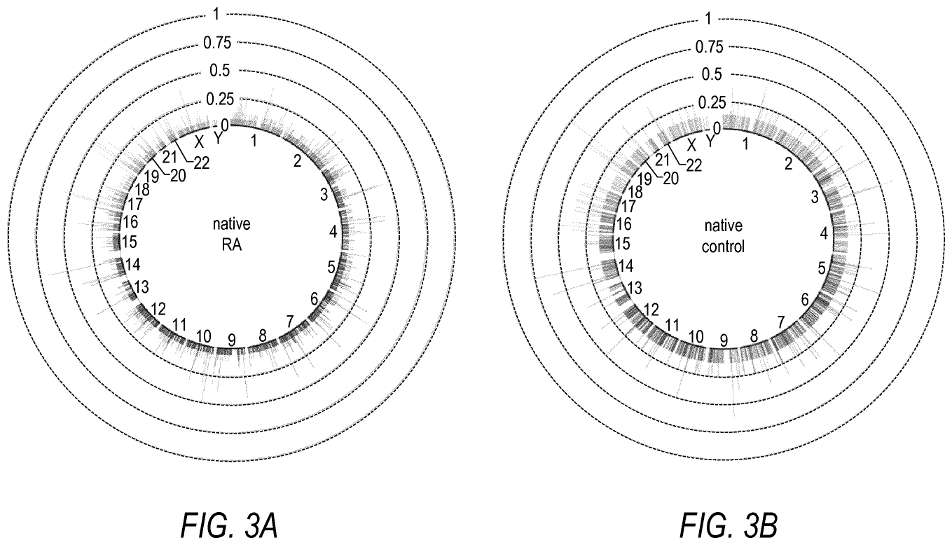

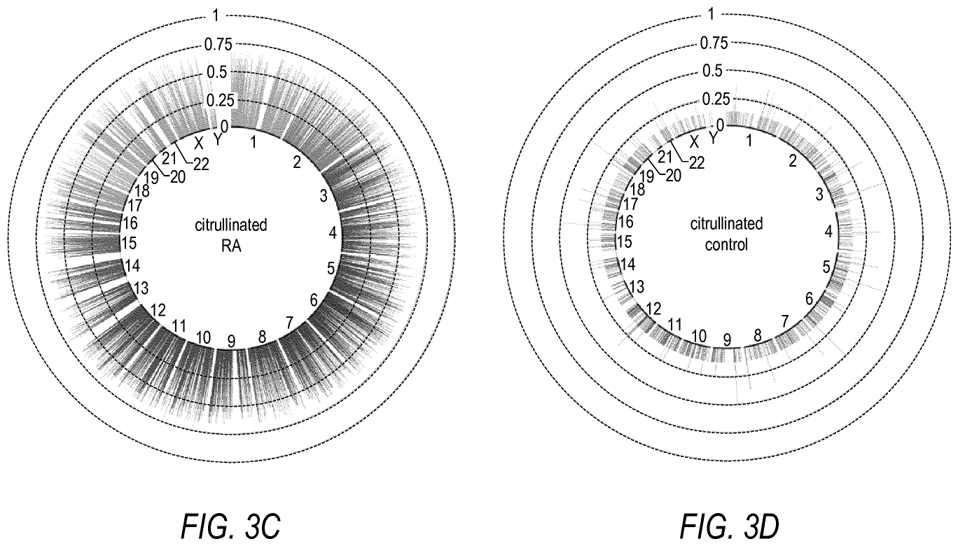

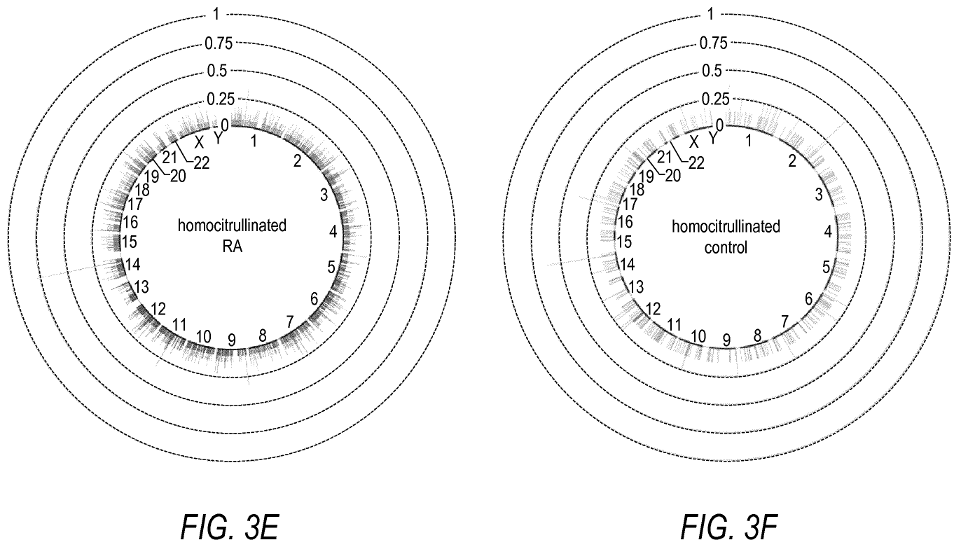

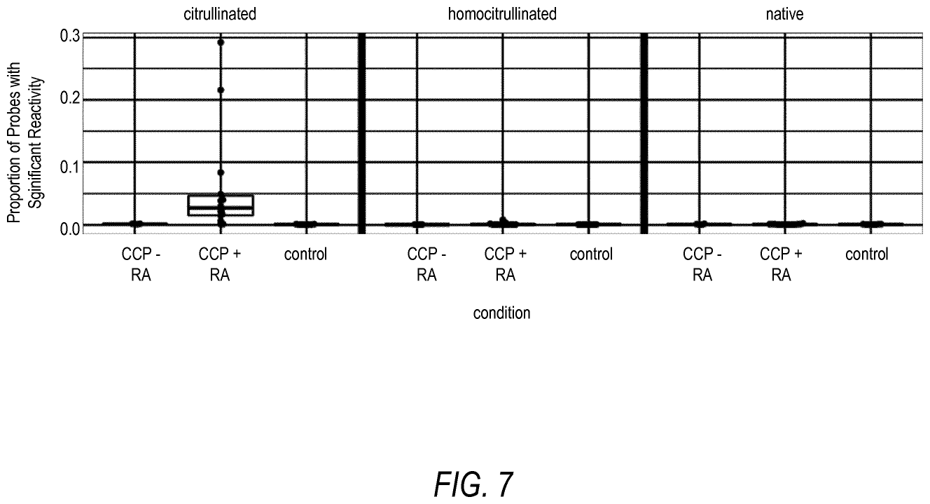

The comprehensive human proteome profiling of antibody reactivity against the native, citrullinated, and homocitrullinated linear peptide probes represents the first unbiased portrait of the RA serum antibody repertoire, which is here termed the RA Abinome. Antibody reactivity against citrullinated peptide probes was detected as described herein at high frequency within the cohort of 18 RA serum samples as attested by the overall heights of the bars throughout the proteome when comparing between the RA group vs. the control group (FIGS. 3A-3F). A total of 8,981 citrullinated peptide probes (0.66% of total citrullinated probes) had significant reactivity present at a frequency of greater than or equal to 10 out of 18 RA samples and potentially represent elements for use in a diagnostic classifier comparable to current CCP2 based diagnostic tests (Arthritis Res Ther 2017; 19:115). This is in contrast to antibody reactivity for native and homocitrullinated peptide probes, which was observed at a substantially lower frequency. Only 4 native peptide probes (SLKRLTDKEADEYYMR (SEQ ID NO: 9), LTDKEADEYYMRRRHL (SEQ ID NO: 10), SQSSPEFKGSLASLSD (SEQ ID NO: 11), and PEFKGSLASLSDSLGV (SEQ ID NO: 12)) and 2 homocitrullinated peptide probes (SQSSPEFJGSLASLSD (SEQ ID NO: 13) and PEFJGSLASLSDSLGV (SEQ ID NO: 14)) showed significant reactivity at a frequency of greater or equal to 10 out of 18 RA samples (SEQ ID NOS: 1-8861). When compared to the 8 control serum samples that were also profiled, the mean number of peptide probes with reactivity between groups were not statistically different for native and homocitrullinated peptide probes (p-values 0.997 and 0.997 respectively). On the other hand, a difference was detected for citrullinated peptide probes (p-value 0.020, FIG. 7 and Table 3).

TABLE 3

| | Citrullinated | | Homocitrullinated | | Native | |
|---|---|---|---|---|---|---|
| | | Std. | | Std. | | Std. |
| | Avg. | Dev. | Avg. | Dev. | Avg. | Dev. |
| CCP positive | 80955.2 | 113707.1 | 1560.2 | 2679.3 | 1023.7 | 831.9 |
| RA | | | | | | |
| CCP negative | 1337.8 | 175.6 | 119.8 | 83.2 | 1095.5 | 772.4 |
| RA | | | | | | |
| Control | 531.0 | 244.5 | 152.0 | 67.7 | 912.0 | 445.9 |

Table 3 shows the average number of significant probes detected by condition organized by substitution category as well as the standard deviation. When comparing between RA (both CCP positive and CCP negative) and controls, the mean number of peptide probes with reactivity between groups were significant for citrullinated peptide probes (p-value 0.020 by homoscedastic t-test) and not significant in native and homocitrullinated peptide probes (p-values 0.997 and 0.997).

Interestingly, antibody reactivity against native peptide probes was observed for control samples, which is consistent with reports by Nagele et al. (PLoS One 2013; 8:e60726). Surprisingly, reactivity against citrullinated and homocitrullinated peptide probes was also observed for control samples and was not restricted to specific individuals. In fact, specific citrullinated and homocitrullinated peptide probes showed reactivity in up to 50% of control individuals but were absent in RA individuals.

In addition to detecting antibody reactivity against citrullinated peptide probes in previously reported antigens such as filaggrin and fibrin, the majority of antibody reactivity detected in RA serum samples against the citrullinated proteome has not been previously reported to be associated with RA (Table 4). Citrullinated peptide probes as listed by UniProt identifier (UniProt) within 340 proteins had antibody reactivity at a frequency (#) greater or equal to 10 out of 18 in RA samples in this cohort (Table 5). The top 25 proteins with the highest frequency of reactivity within this RA cohort are presented in Table 6.

TABLE 4
Frequency of reactivity to commonly known antigens

| | | Proportion of RA samples | | |
|---|---|---|---|---|
| | | with Reactivity | | |
| Antigen | UniProt | AA | C | H |
| Filaggrin | P20930 | 0.06 | 0.61 | 0.06 |
| Fibrin | | | | |
| Fibrinogen alpha | P02671 | 0.00 | 0.61 | 0.06 |
| chain | | | | |
| Fibrinogen beta chain | P02675 | 0.00 | 0.56 | 0.06 |
| Fibrinogen gamma | P02679 | 0.00 | 0.17 | 0.00 |
| chain | | | | |
| BiP | P11021 | 0.00 | 0.44 | 0.06 |
| RA33 | P22626 | 0.17 | 0.67 | 0.00 |
| Vimentin | P08670 | 0.06 | 0.39 | 0.00 |
| alpha-enolase | P06733 | 0.00 | 0.33 | 0.00 |
| PAD4 | Q9UM07 | 0.00 | 0.61 | 0.00 |

| | | Proportion of Control samples | | |
|---|---|---|---|---|
| | | with Reactivity | | |
| Antigen | UniProt | AA | C | H |
| Filaggrin | P20930 | 0.00 | 0.00 | 0.00 |
| Fibrin | | | | |
| Fibrinogen alpha | P02671 | 0.00 | 0.00 | 0.00 |
| chain | | | | |
| Fibrinogen beta chain | P02675 | 0.00 | 0.00 | 0.00 |
| Fibrinogen gamma | P02679 | 0.00 | 0.00 | 0.00 |
| chain | | | | |
| BiP | P11021 | 0.00 | 0.00 | 0.00 |
| RA33 | P22626 | 0.25 | 0.13 | 0.00 |
| Vimentin | P08670 | 0.13 | 0.00 | 0.00 |
| alpha-enolase | P06733 | 0.00 | 0.00 | 0.00 |
| PAD4 | Q9UM07 | 0.00 | 0.00 | 0.00 |

AA: Autoantibody (Native);
C: Citrullinated;
H: homocitrullinated

TABLE 5

| UniProt | # |
|---|---|
| P09848 | 14 |
| Q15399 | 14 |
| O94919 | 14 |
| Q9Y5H2 | 14 |
| Q9Y2C9 | 14 |
| P51841 | 13 |
| Q9Y5Y9 | 13 |
| Q9BXJ2 | 13 |
| P40121 | 13 |
| O75077 | 13 |
| Q9Y6Z7 | 13 |
| Q8NHL6 | 13 |
| Q5VUB5 | 13 |
| P47901 | 12 |
| P0C0L4 | 12 |
| P23435 | 12 |
| P10767 | 12 |
| Q02846 | 12 |
| P14735 | 12 |
| P56199 | 12 |
| Q15046 | 12 |
| O60928 | 12 |
| P32004 | 12 |
| Q15113 | 12 |
| P43119 | 12 |
| Q99835 | 12 |
| Q9UP52 | 12 |
| Q13093 | 12 |
| Q7L0X0 | 12 |
| Q13421 | 12 |
| Q9NZU1 | 12 |
| O94991 | 12 |
| Q8N0Z9 | 12 |
| Q9UN66 | 12 |
| Q9BYF1 | 12 |
| Q9HCQ7 | 12 |
| Q6UWM9 | 12 |
| Q9H0X4 | 12 |
| Q9NT22 | 12 |
| Q8WUJ1 | 12 |
| Q8NGT5 | 12 |
| Q86SJ6 | 12 |
| Q86VR7 | 12 |
| Q8N158 | 12 |
| Q5UCC4 | 12 |
| P0C8F1 | 12 |
| Q8IWL1 | 12 |
| P0C0L5 | 12 |
| P01185 | 12 |
| P01732 | 12 |
| P13569 | 12 |
| Q14118 | 12 |
| P35462 | 12 |
| P29122 | 12 |
| Q9UBN4 | 12 |
| O00294 | 12 |
| O43820 | 12 |
| Q8N2S1 | 12 |
| O95460 | 12 |
| O95838 | 12 |
| Q92544 | 12 |
| O43561 | 12 |
| Q9NZP8 | 12 |
| Q96DB9 | 12 |
| Q6UY14 | 12 |
| Q86XS8 | 12 |
| Q9Y5G1 | 12 |
| Q6UXY8 | 12 |
| Q8N614 | 12 |
| Q9BXR5 | 12 |
| Q9C0A0 | 12 |
| Q9BWV1 | 12 |
| Q8IWL2 | 12 |
| O43306 | 11 |
| Q16671 | 11 |
| P02749 | 11 |
| Q9UL51 | 11 |
| P04003 | 11 |
| P32970 | 11 |
| P29323 | 11 |
| O75487 | 11 |
| Q9BTY2 | 11 |
| P22352 | 11 |
| O15303 | 11 |
| P12544 | 11 |
| P01574 | 11 |
| P29460 | 11 |
| P06858 | 11 |
| Q14767 | 11 |
| Q99467 | 11 |
| P55082 | 11 |
| P08138 | 11 |
| Q96S42 | 11 |
| O00634 | 11 |
| P55058 | 11 |
| Q92743 | 11 |
| P14672 | 11 |
| Q9NY91 | 11 |
| P47989 | 11 |
| O43506 | 11 |
| O43320 | 11 |
| Q8NHW4 | 11 |
| Q9NR23 | 11 |
| Q9BQT9 | 11 |
| O43291 | 11 |
| Q9Y581 | 11 |
| Q7L8A9 | 11 |
| Q9Y2G8 | 11 |
| Q96F46 | 11 |
| Q9UJQ1 | 11 |
| Q86VR8 | 11 |
| Q16769 | 11 |
| Q8IUX8 | 11 |
| Q9NYZ4 | 11 |
| O43692 | 11 |
| Q9NP84 | 11 |
| Q6AZY7 | 11 |
| Q9NR61 | 11 |
| Q9C0C4 | 11 |
| Q9NZD1 | 11 |
| Q9NPH9 | 11 |
| Q9Y5F8 | 11 |
| Q96SM3 | 11 |
| Q9NR82 | 11 |
| Q9HCU0 | 11 |
| Q9GZU1 | 11 |
| Q6UXK2 | 11 |
| Q9H237 | 11 |
| Q9BUR5 | 11 |
| Q5FYB0 | 11 |
| Q9H6B4 | 11 |
| Q96PJ5 | 11 |
| Q9H0B8 | 11 |
| Q96AP7 | 11 |
| Q8WXF3 | 11 |
| Q8NH00 | 11 |
| Q8NGU2 | 11 |
| Q96KN9 | 11 |
| Q8NET1 | 11 |
| Q30KQ7 | 11 |
| Q86XS5 | 11 |
| Q6P1S2 | 11 |
| Q6ZWJ8 | 11 |
| Q96R09 | 11 |
| Q69YU5 | 11 |
| Q04771 | 11 |
| P35348 | 11 |
| P18089 | 11 |
| P49913 | 11 |
| P21554 | 11 |
| P13942 | 11 |
| P29320 | 11 |
| P54764 | 11 |
| P54756 | 11 |
| O15197 | 11 |
| P00742 | 11 |
| Q12805 | 11 |
| P55259 | 11 |
| P08238 | 11 |
| Q12794 | 11 |
| P48357 | 11 |
| Q9ULZ9 | 11 |
| Q99102 | 11 |
| P13591 | 11 |
| P00973 | 11 |
| P78380 | 11 |
| P08183 | 11 |
| P29622 | 11 |
| P16109 | 11 |
| P05452 | 11 |
| P41587 | 11 |
| O75882 | 11 |
| O14788 | 11 |
| O14494 | 11 |
| Q9H013 | 11 |
| P50591 | 11 |
| Q9UNQ0 | 11 |
| Q14667 | 11 |
| Q9Y6N6 | 11 |
| O95399 | 11 |
| Q9UKP5 | 11 |
| Q9ULW2 | 11 |
| Q9BZP6 | 11 |
| Q9NNX6 | 11 |
| Q8N608 | 11 |
| Q8WWQ2 | 11 |
| Q9BZ11 | 11 |
| Q9H0U3 | 11 |
| Q6UWL6 | 11 |
| Q8NA29 | 11 |
| Q96JP9 | 11 |
| Q96P20 | 11 |
| Q96CH1 | 11 |
| Q6P531 | 11 |
| Q8TDQ1 | 11 |
| Q8NBM4 | 11 |
| Q6T423 | 11 |
| B8ZZ34 | 11 |
| P02771 | 10 |
| P43251 | 10 |
| O60840 | 10 |
| P06126 | 10 |
| P11912 | 10 |
| O15335 | 10 |
| P24855 | 10 |
| Q07507 | 10 |
| P25101 | 10 |
| P98172 | 10 |
| Q15768 | 10 |
| Q15375 | 10 |
| P54762 | 10 |
| P03951 | 10 |
| P12314 | 10 |
| P36888 | 10 |
| P40197 | 10 |
| O43424 | 10 |
| P05111 | 10 |
| P08514 | 10 |
| P26006 | 10 |
| P17658 | 10 |
| Q9UNX9 | 10 |
| P09382 | 10 |
| Q01718 | 10 |
| P09238 | 10 |
| P33527 | 10 |
| Q93086 | 10 |
| P47900 | 10 |
| Q16557 | 10 |
| P49190 | 10 |
| P35542 | 10 |
| P22732 | 10 |
| Q9UPR5 | 10 |
| P20062 | 10 |
| P16035 | 10 |
| O00295 | 10 |
| O00744 | 10 |
| P61073 | 10 |
| Q9NPG1 | 10 |
| Q13477 | 10 |
| Q9Y5G5 | 10 |
| Q92478 | 10 |
| Q9Y258 | 10 |
| P59901 | 10 |
| Q7Z4F1 | 10 |
| O60412 | 10 |
| Q9UBP4 | 10 |
| Q9Y336 | 10 |
| Q9NYY1 | 10 |
| Q4KMG0 | 10 |
| Q9H228 | 10 |
| Q8IWV1 | 10 |
| Q9NRA1 | 10 |
| Q9HD89 | 10 |
| Q96GW7 | 10 |
| Q9GZN4 | 10 |
| Q32P28 | 10 |
| Q96QV1 | 10 |
| Q9BSN7 | 10 |
| Q99944 | 10 |
| Q8WTR4 | 10 |
| Q96JB6 | 10 |
| Q9H5Y7 | 10 |
| Q96KN2 | 10 |
| Q3SXP7 | 10 |
| Q9BU40 | 10 |
| Q969P0 | 10 |
| Q96PL5 | 10 |
| Q96L15 | 10 |
| Q9BQY6 | 10 |
| Q8J025 | 10 |
| Q53RT3 | 10 |
| Q8TCU3 | 10 |
| Q7RTT9 | 10 |
| P59535 | 10 |
| Q641Q3 | 10 |
| A6NLU5 | 10 |
| Q6DN72 | 10 |
| A6NI73 | 10 |
| Q7Z410 | 10 |
| Q6AI14 | 10 |
| Q8NGD1 | 10 |
| Q5T1S8 | 10 |
| Q6MZM9 | 10 |
| Q5JRS4 | 10 |
| Q6UX82 | 10 |
| Q8N967 | 10 |
| P28039 | 10 |
| P23634 | 10 |
| P23560 | 10 |
| O00238 | 10 |
| P28908 | 10 |
| Q12884 | 10 |
| P41439 | 10 |
| P28472 | 10 |
| O14764 | 10 |
| P55107 | 10 |
| P36382 | 10 |
| Q99679 | 10 |
| P48058 | 10 |
| P17693 | 10 |
| P08887 | 10 |
| P48549 | 10 |
| P31025 | 10 |
| P29376 | 10 |
| P61626 | 10 |
| P55083 | 10 |
| P34130 | 10 |
| Q9UN70 | 10 |
| P60201 | 10 |
| P49862 | 10 |
| Q92932 | 10 |
| Q99731 | 10 |
| P23975 | 10 |
| P48067 | 10 |
| Q12846 | 10 |
| Q14515 | 10 |
| Q14162 | 10 |
| Q15403 | 10 |
| Q99985 | 10 |
| Q9UKF2 | 10 |
| O94956 | 10 |
| Q9Y5E4 | 10 |
| O15204 | 10 |
| Q9UHX3 | 10 |
| Q8WU66 | 10 |
| Q8IWT6 | 10 |
| Q9P0V8 | 10 |
| Q96N19 | 10 |
| Q9P2E7 | 10 |
| Q9HB40 | 10 |
| Q9H239 | 10 |
| Q8NC67 | 10 |
| Q8TAA9 | 10 |
| Q6UWQ5 | 10 |
| Q7Z4W2 | 10 |
| Q86Z14 | 10 |
| Q8NET8 | 10 |
| Q6QNK2 | 10 |
| Q8NGU9 | 10 |

TABLE 6
25 most frequent citrullinated proteins associated with RA

| UniProt | | Proportion of RA | Previous Association |
|---|---|---|---|
| ID | Gene | patients with reactivity | with RA |
| P09848 | LCT | 0.778 | No |
| Q15399 | TLR1 | 0.778 | No |
| O94919 | ENDOD1 | 0.778 | No |
| Q9Y5H2 | PCDHGA11 | 0.778 | No |
| Q9Y2C9 | TLR6 | 0.778 | No |
| P51841 | GUCY2F | 0.722 | No |
| Q9Y5Y9 | SCN10A | 0.722 | No |
| Q9BXJ2 | C1QTNF7 | 0.722 | No |
| P40121 | CAPG | 0.722 | Yes |
| O75077 | ADAM23 | 0.722 | No |
| Q9Y6Z7 | COLEC10 | 0.722 | No |
| Q8NHL6 | LILRB1 | 0.722 | Yes |
| Q5VUB5 | FAM171A1 | 0.722 | No |
| P47901 | AVPR1B | 0.667 | No |
| P0C0L4 | C4A | 0.667 | No |
| P23435 | CBLN1 | 0.667 | No |
| P10767 | FGF6 | 0.667 | No |
| Q02846 | GUCY2D | 0.667 | No |
| P14735 | IDE | 0.667 | No |
| P56199 | ITGA1 | 0.667 | No |
| Q15046 | KARS | 0.667 | No |
| O60928 | KCNJ13 | 0.667 | No |
| P32004 | L1CAM | 0.667 | No |
| Q15113 | PCOLCE | 0.667 | No |
| P43119 | PTGIR | 0.667 | Yes |

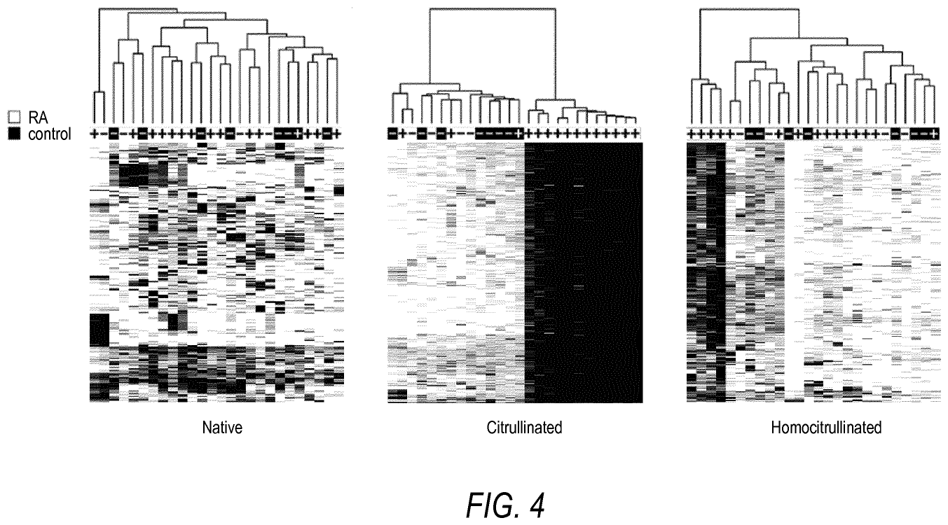

Within the RA Abinome, hierarchical clustering analysis showed 12 out of 18 RA samples had consistent peptide level reactivity against citrullinated peptide probes and readily formed a distinctive group of RA patients from controls (FIG. 4, center). In contrast, antibody reactivity against native peptide probes (autoantibody) did not show any ability to distinguish between RA and control samples (FIG. 4, left). Interestingly, strong reactivity against homocitrullinated peptide probes was seen in four RA samples, although those individuals also showed reactivity against citrullinated peptide probes and did not constitute a homocitrulline positive, citrulline negative subgroup (FIG. 4, right).

The nanoliter-scale immunoassay platform was used to validate the peptide array findings in RA sera. Due to the overlapping nature of the linear peptide array, antibody reactivity against a linear epitope is expected to be represented by signal from multiple contiguous peptide probes. As these epitopes may have potential implications for RA prognosis/diagnosis and are the bases of current diagnostic assays, frequently occurring citrullinated epitopes observed in RA samples are summarized in Table 7. Consistent with Table 6, the majority of the proteins with frequently occurring epitopes has not been previously associated with RA. While summarizing RA specific epitopes represents a more restrictive view, RA specific antibody reactivity at over 50% frequency was nonetheless observed in this cohort. These frequently occurring epitopes against citrullinated peptide probes range from 20 amino acids (2 contiguous peptides) in size to as long as 44 amino acids (7 contiguous peptides), with a mean of 26.7 amino acids. Frequently occurring epitopes at 50% frequency were not seen against homocitrullinated and native peptides exclusively in RA serum samples, nor were these observed in control samples.

TABLE 7
25 most frequent citrullinated epitopes associated with
RA. Start and end positions refer to the first and last
positions of the peptides found within the epitope.

| UniProt | | | | Mean Proportion of RA |
|---|---|---|---|---|
| ID | Gene | Start | End | Patients with Reactivity |
| P0C8F1 | PATE4 | 29 | 68 | 0.57 |
| Q69YU5 | C12orf73 | 33 | 56 | 0.56 |
| Q8WXF3 | RLN3 | 89 | 128 | 0.57 |
| Q8NET1 | DEFB108B | 13 | 32 | 0.56 |
| Q9UMX5 | NENF | 69 | 112 | 0.58 |
| Q30KQ7 | DEFB113 | 33 | 52 | 0.56 |
| Q8NHW4 | CCL4L2 | 61 | 80 | 0.56 |
| Q99731 | CCL19 | 57 | 76 | 0.56 |
| P0DJI8 | SAA1 | 49 | 72 | 0.61 |
| P0DJI9 | SAA2 | 49 | 72 | 0.61 |
| Q96NZ9 | PRAP1 | 69 | 96 | 0.56 |
| Q9NP84 | TNFRSF12A | 29 | 52 | 0.56 |
| P49913 | FALL39 | 105 | 132 | 0.57 |
| Q9NPH9 | IL26 | 21 | 48 | 0.56 |
| P78380 | OLR1 | 217 | 260 | 0.57 |
| P35542 | SAA4 | 49 | 68 | 0.56 |
| Q9BQY6 | WFDC6 | 57 | 76 | 0.56 |
| P01185 | AVP | 13 | 36 | 0.64 |
| Q9P0M4 | IL17C | 41 | 68 | 0.58 |
| Q9NYY1 | IL20 | 41 | 64 | 0.56 |
| Q99470 | SDF2 | 65 | 92 | 0.64 |
| P01574 | IFNB1 | 121 | 144 | 0.56 |
| P32970 | CD70 | 153 | 176 | 0.56 |
| Q9HCQ7 | NPVF | 37 | 60 | 0.63 |
| A8K4G0 | UNQ2530/PRO6029 | 53 | 76 | 0.56 |

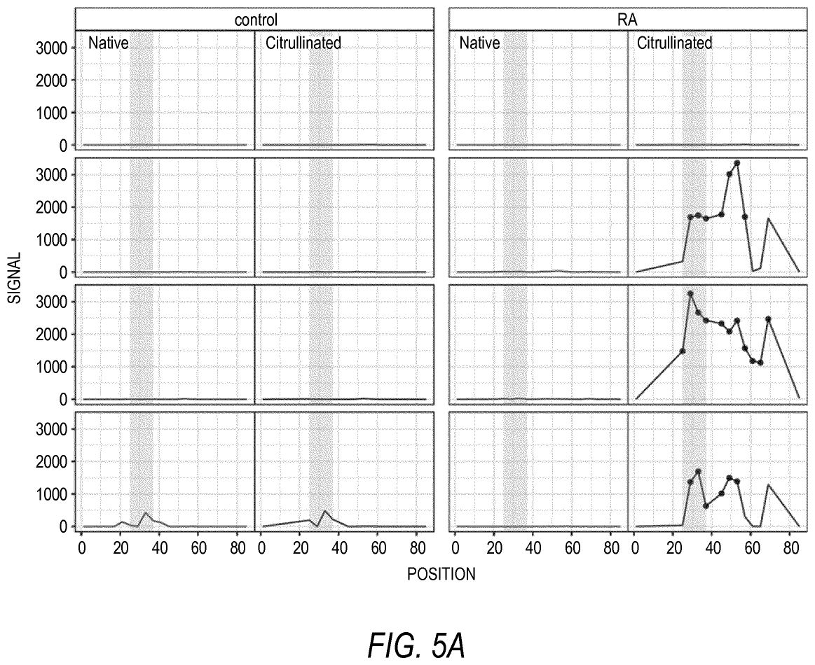

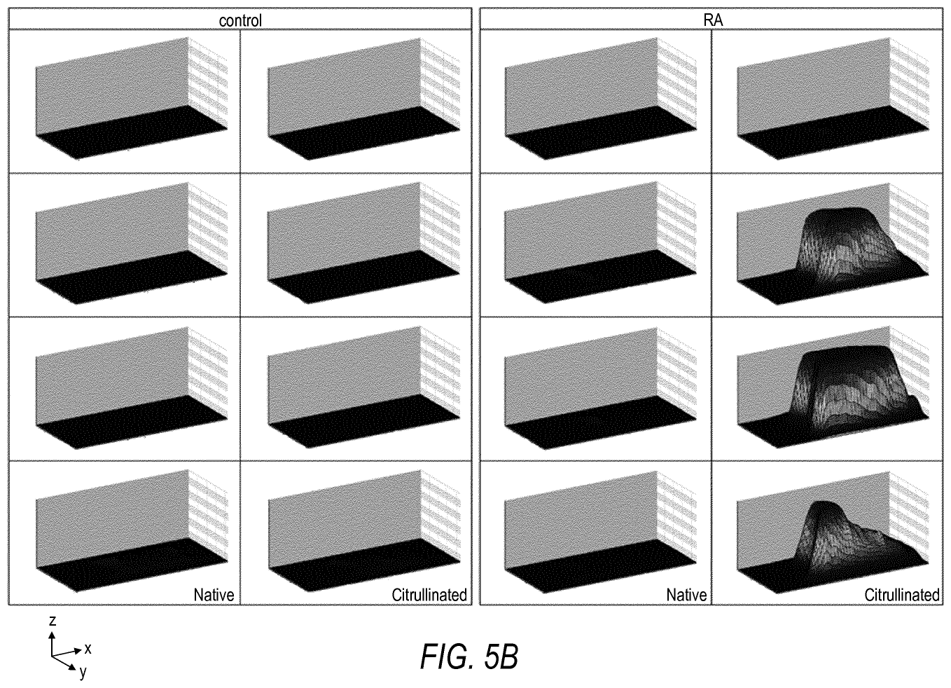

To verify antibody reactivity findings for frequently occurring epitopes, the nanoliter-scale immunoassay system was used. The nanoliter-scale immunoassay system employs microfluidics to carry out an automated miniature ELISA assay. An illustrative example is shown in FIGS. 5A and 5B, where peptide sequence CNTCIYTEGWKCMAG-R/cit-GTCIAKENELCS (SEQ ID NO: 1) (shaded area centered about position 30) corresponding to positions 29-56 of the protein PATE4 (POCRF1) and including four contiguous peptide probes on array, were synthesized commercially as a single epitope with C-terminal biotinylation via a terminal lysine residue. In all eight samples shown in FIGS. 5A and 5B (four controls and four RA samples), little to no detectable antibody reactivity was observed for the native peptides on array. This is consistent with integrated signal obtained from the nanoliter-scale immunoassay system. In contrast, three out of four RA samples showed strong reactivity on array for the citrullinated peptides within this epitope, as well as the nanoliter-scale immunoassay system (FIG. 5B, far right column). While the overall concordance between the signal generated from the nanoliter-scale immunoassay system and the disclosed peptide array is high, due to differences in surface properties between the peptide array surface and the nanoliter-scale immunoassay system streptavidin coated beads, signal between the two technologies cannot be quantitatively reproduced perfectly (i.e., by regression coefficients and R² values). However, a qualitative assessment comparing the array signal and the nanoliter-scale immunoassay system output for the 20+ epitopes in the validation experiment showed concordance 86.4% of the time.

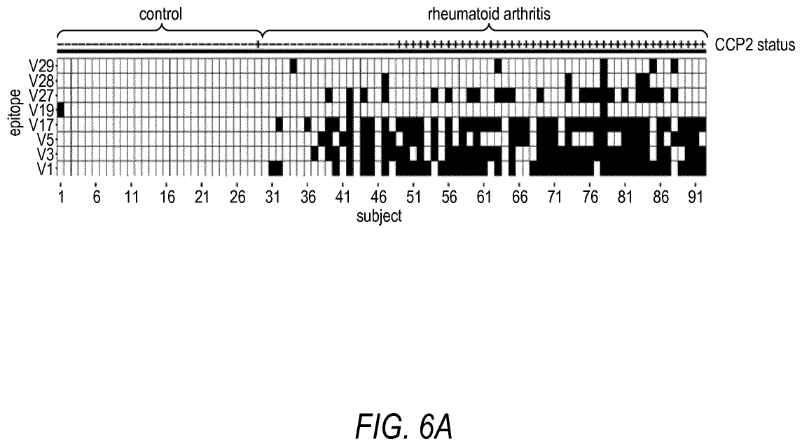

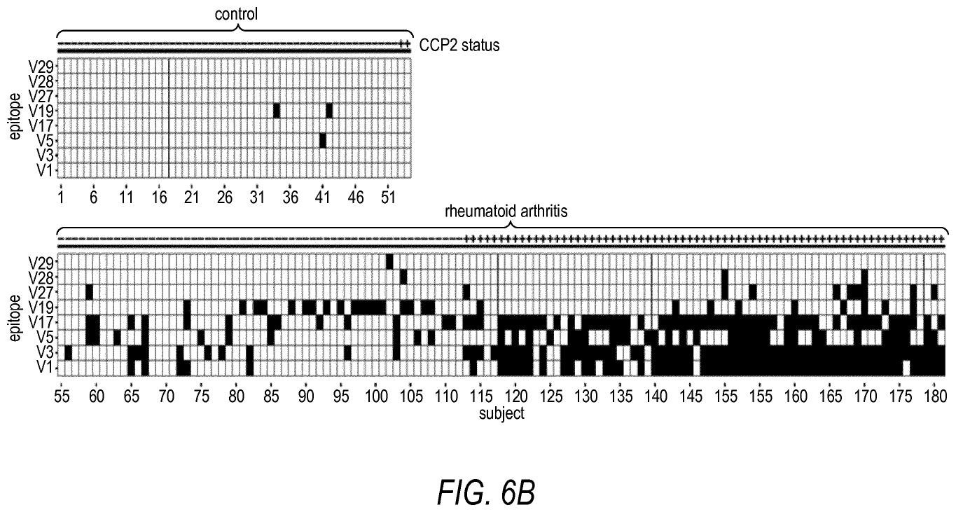

Given the large number of frequently occurring citrullinated epitopes as shown in Table 7, the possibility of constructing a novel RA diagnostic classifier based on epitope data derived from the peptide array using the nanoliter-scale immunoassay system was explored. As shown in FIGS. 6A and 6B, overall diagnostic performance of an 8-epitope classifier (sequences shown in Table 1) was first assessed on a cohort of 92 samples (29 controls and 63 RA samples), yielding 96% specificity and 92% sensitivity. As a comparison, the present RA samples were tested using a commercially available CCP2 assay, yielding 96.6% specificity and 68.3% sensitivity. Selection of the 8-eptiope classifier was achieved by selecting epitopes that collectively resulted in a positive identification of each of those subject samples characterized as being positive RA while ensuring that control (i.e., RA negative) samples were not identified. Considering, for example, RA positive samples 50-60 only, epitope V1 provided a positive identification for RA samples 50-53 and 55-60, while successfully distinguishing control samples (i.e., control samples were not identified as RA positive). Accordingly, to identify RA positive sample 54, several epitopes could be selected for use in combination with epitope, including at least epitopes V3, V5, V17, and V27. Notably, each of epitopes V3, V5, V17, and V27 additionally provided for redundant (unique) or non-redundant (non-unique) identification of other RA positive samples as see in FIG. 6A.

It will be appreciated that a variety of approaches may be taken to select a combination of epitopes for use as a peptide classifier. If qualitative or quantitative information is known about the ability of an epitope to identify one or more RA positive samples from a set of samples while also successfully distinguishing control samples, then a combination of epitopes can be selected by hand or with automated methods that collectively and correctly identify at least a majority of a set of samples. Automated methods include machine learning algorithms as described herein, and other automated methods for assembling a combination of epitopes as will be appreciated by one of ordinary skill in the art. In certain situations, it may be valuable to gauge the efficacy of a set of epitopes for use as a peptide classifier by measuring or calculating the sensitivity and specificity of the set of epitopes. At present, leading CCP2 assays are capable of delivering a specificity of about 0.95 and a sensitivity of about 0.70. Therefore, it may be useful to select a peptide classifier with a specificity of at least 0.95 and a sensitivity of at least 0.70.

To further validate the diagnostic performance of the disclosed 8-epitope classifier (Table 1), an independent cohort of 181 serum samples (54 controls and 127 RA samples) was obtained from a commercial source and tested on the nanoliter-scale immunoassay system (FIG. 6B). The overall diagnostic performance in the validation cohort was 95% specificity and 85% sensitivity.

1. A composition, comprising:
a plurality of molecules, each molecule comprising a peptide having a sequence selected from SEQ ID NOS: 1-8861,
wherein the plurality of molecules defines a classifier for rheumatoid arthritis.

2. The composition of claim 1, wherein the classifier discriminates between a sample derived from a first population and a sample derived from a second population.

3. The composition of claim 2, wherein the first population is defined by subjects having at least one marker associated with a first disease state and the second population is defined by subjects lacking the at least one marker associated with the first disease state.

4. The composition of claim 3, wherein the first disease state is rheumatoid arthritis.

5. The composition of claim 3, wherein the marker associated with a first disease state is one of an antibody and a serum marker.

6. The composition of claim 1, wherein the classifier discriminates between a sample derived from a first population and a sample derived from a second population,
wherein the sample derived from the first population comprises at least one marker associated with a first disease state and the sample derived from the second population lacks the at least one marker associated with the first disease state.

7. The composition of claim 1, wherein the classifier discriminates between a sample derived from a first population and a sample derived from a second population,
wherein at least one marker associated with a first disease state is present in the sample derived from the first population and wherein the at least one marker is absent in the sample derived from the second population.

8. The composition of claim 3, wherein the marker is one of an anti-citrullinated peptide antibody, and anti-homocitrullinated peptide antibody, an autoantibody, an anti-cyclic citrullinated peptide antibody, and an anti-cyclic homocitrullinated peptide antibody.

9. The composition of claim 1, wherein the plurality of molecules comprises at least 3 different molecules.

10. The composition of claim 1, wherein the classifier distinguishes between a sample derived from a first group defined by a first disease state and a sample derived from a second group defined by a second disease state.

11. The composition of claim 10, wherein the first group is defined by subjects having a positive diagnosis for rheumatoid arthritis.

12. The classifier of claim 1, wherein the synthetic classifier is one of a diagnostic classifier and a prognostic classifier.

13. The composition of claim 1, wherein the plurality of molecules comprises peptides having a sequence selected from a first list, the first list consisting of the sequences listed in Table 1 (SEQ ID NOS: 1-8).

14. A peptide classifier, comprising:
a plurality of molecules, each molecule comprising a peptide having a sequence selected from SEQ ID NOS: 1-8861, the molecules representing at least 3 different sequences selected from SEQ ID NOS: 1-8861.

15. A method for identifying the presence of an antibody indicative of rheumatoid arthritis in a sample, the method comprising:
contacting a sample derived from a subject with a composition, the composition comprising a plurality of molecules, each molecule comprising a peptide having a sequence selected from SEQ ID NOS: 1-8861, the molecules representing at least 3 different sequences selected from SEQ ID NOS: 1-8861;
binding an antibody present in the sample to at least one of the plurality of molecules, thereby forming an antibody-peptide conjugate; and
detecting the antibody-peptide conjugate, thereby identifying the presence of the antibody in the sample.

16. A kit for identifying the presence of an antibody indicative of rheumatoid arthritis in a sample, the kit comprising:
a solid support having a plurality of molecules bound thereon, each molecule comprising a peptide having a sequence selected from SEQ ID NOS: 1-8861, the molecules representing at least 3 different sequences selected from SEQ ID NOS: 1-8861; and
a detectable antibody to a human antibody.

17. The kit of claim 24, wherein the human antibody is one of an anti-citrullinated peptide antibody, and anti-homocitrullinated peptide antibody, an autoantibody, and an anti-cyclic citrullinated peptide antibody.

18. A device for identifying the presence of an antibody indicative of rheumatoid arthritis in a sample, the device comprising:
A solid support or a flow cell having a plurality of molecules bound therein, each molecule comprising a peptide having a sequence selected from SEQ ID NOS: 1-8861, the molecules representing at least 3 different sequences selected from SEQ ID NOS: 1-8861, said solid support or flow cell capable of receiving a sample derived from a subject at the location of the plurality of molecules.
